(19) United States
(12) Patent Application Publication
(10) Pub. No.: US 2026/0069770 A1
(43) Pub. Date: Mar. 12, 2026

(54) DRIVE MECHANISM FOR LARGE VOLUME DELIVERY DEVICE

(71) Applicant: Sanofi-Aventis Deutschland GmbH, Frankfurt am Main (DE)

(72) Inventors: Marc Anderson, Marblehead, MA (US); Donald Ziniti, Cumberland, RI (US)

(21) Appl. No.: 18/826,573

(22) Filed: Sep. 6, 2024

(51) Int. Cl. A61M 5/145; A61M 5/14

(52) U.S. Cl. CPC A61M 5/1456; A61M 5/1413

(57) ABSTRACT

A system for delivering a medicament is provided. The system can include a drive mechanism configured to drive a plunger of a medicament container. The medicament container may be contained in a container sled, and the drive mechanism can be securely held by a drive holder adjacent the container sled. The drive holder can be secured within an external housing of the system allowing the container to move with the container sled to form a connection with a fluid delivery pathway.

The present disclosure relates generally to devices and methods for providing a drive mechanism in an medicament delivery device, and more specifically for a large volume delivery device.

Injection devices may be used to deliver a fluid containing a pharmaceutical drug or medicament to a patient. For example, the medicament may be delivered by the injection device to the patient via a needle, cannula, tube, microneedle array, or other route.

One type of injector used to provide such a medicament is a large volume device (LVD), which may also be known as a bolus injector or a reservoir-type injector. A large volume device may provide a relatively large volume of medicament, typically at least 1 mL or more. The large volume device is generally positioned against the skin or held to the skin at a suitable injection site, and upon activation of the device, medicament is injected through the patient's skin.

The medicament must be provided to the injection device and made accessible so that the injection device can deliver the medicament to the patient. A drive mechanism is engaged to force a medicament out of a container. In some cases, the drive mechanism creates a significant force to push the medicament through a fluid path and into a needle or cannula inserted in a patient's body. Depending on the configuration of the various components of the delivery device, the force exerted by the drive mechanism must be counteracted so that the force generated by the drive mechanism is transmitted towards a plunger of the vial or cartridge. However, in order for the drive to appropriately transmit force with certain more complicated device configurations, additional components are needed to secure the drive in place and allow appropriate assembly.

Accordingly, the present disclosure provides devices with improved methods for assembling and securing the drive mechanism to allow appropriate function of certain medicament delivery systems.

### SUMMARY

According to certain embodiments, a system for delivering medicament is provided. The system can include an external housing, a medicament container sled within the external housing, a drive holder, a medicament container comprising a plunger and an internal volume to hold medicament, and a drive mechanism. The drive mechanism can be configured to enter the driver holder and to be securely retained in the drive holder.

In another embodiment, a drive mechanism is provided. The drive mechanism can include a drive housing. The drive housing can include at least one ridge, at least one protrusion, and a pair of walls. The drive mechanism can include a drive release latch, and a piston mechanism including a piston, a drive outer sleeve, and a detent. The pair of walls can be configured to interact with the drive release latch. The drive mechanism can be configured to fit into a drive holder of an external housing.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1A is cross-sectional view of a large volume injection device.

FIG. 1B is perspective view of a large volume injection device

FIGS. 2A and 2B are perspective views illustrating a drive mechanism in accordance with one or more embodiments.

FIG. 3 is a perspective view illustrating assembly of the drive mechanism from one perspective.

FIG. 4 is a perspective view illustrating an assemble view of the drive mechanism in accordance with one or more embodiments.

FIG. 5 is a perspective view illustrating additional features of a drive mechanism with a drive holder, according to various embodiments.

FIG. 6A-6C are perspective views of components of a delivery device including a rear opening for insertion of a drive mechanism in accordance with one or more embodiments.

FIGS. 7A and 7B are perspective views illustrating activation of the drive mechanism in accordance with one or more embodiments.

FIGS. 8A-8C are perspective views illustrating an operation flow in accordance with one or more embodiments

FIGS. 9A and 9B are perspective views illustrating activation of an EOD switch mechanism in accordance with one or more embodiments

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purposes.

The presently discussed devices, methods, and systems can be used to load a medicament container into a large volume delivery device in a convenient and cost-effective manner while maintaining a high degree of sanitation for critical components.

The present disclosure is described as providing “aseptic” connections for drug delivery devices. As used herein, “aseptic” or “aseptic condition” will be understood to refer to a condition wherein a device is free of substantially all, but not necessarily all microorganisms such as bacteria, viruses or fungi. As used herein, an “aseptic connection” refers to components that can allow connection of a fluid flow path with a medicament container of a device while preventing introduction of microorganisms. The “connection” need not be already connected but can be capable of forming the connection when ready for use. The terms “aseptic,” “aseptic condition” or “aseptic connection” may not necessarily require that the device be sterile or free of all microorganisms (as sterile may be defined by regulatory requirements), but “aseptic” and “aseptic connection” will be understood to encompass devices that are sterile or maintain sterility. Further, terms “sterile,” “sterile condition,” “sterile connection,” “sterilizing,” or its variations will be understood to include descriptions for the terms “aseptic,” “aseptic condition” and “aseptic connection” and/or very nearly sterile conditions or processes, unless indicated otherwise. Use of the terms “sterile,” “sterile condition,” “sterile connection,” “sterilizing” or its variations will be understood to include use of the terms “aseptic,” “aseptic condition” and “aseptic connection” and/or very nearly sterile conditions or processes, unless indicated otherwise.

Typical injection volumes can range from about 1 mL to over 10 mL. The devices may produce a wide range of injection rates from 0.2 mL/min up to 204.0 mL/min. Such injection profiles may be generally constant in flow rate, generally continuous in duration, or both generally constant and generally continuous. These injections can also occur in a single step of administration. Such injection profiles may be referred to as bolus injections.

Delivery devices functioning with such medicaments may utilize a needle, cannula, or other injection element configured to deliver a medicament to the patient. Such an injection element may, for example, have an external size or diameter of 27 G or less. Further, the injection element could be rigid, flexible, and formed using a range of one or more materials. And in some embodiments, the injection element may include two or more components. For example, a rigid trocar may operate in conjunction with a flexible cannula. Initially, both the trocar and cannula may move together to pierce the skin. The trocar may then retract while the cannula remains at least partially within the target tissue. Later, the cannula may separately retract into the delivery device.

An example delivery device may involve a needle-based injection system as described in ISO 11608-1:2022. Needle-based injection systems may be broadly distinguished into multi-dose container systems and single-dose (with partial or full evacuation) container systems. The container may be a replaceable container or an integrated non-replaceable container.

A multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2022, a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). A single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An insertion mechanism for inserting the needle may take any suitable form. It may be a mechanical spring-based mechanism. Alternatively, the insertion element mechanism may for instance include an electric motor and a gear mechanism that causes insertion of the insertion element into the user. Needle insertion may also be part of a manual action by a user achieved before medicament delivery starts. Alternatively, the insertion mechanism may be a gas or fluid pressure operated mechanism, in which case the needle driving energy source is either a reservoir of pressurized gas or a chemical system in which two or more chemicals are mixed together to produce gas or fluid pressure.

One type of delivery device includes a Large Volume Device (LVD). An LVD delivery device is configured to dispense a relatively large dose of medicament, in particular at least 1 mL and typically up to 2.5 mL, but possibly up to 10 mL. LVDs can also be configured for bolus or basal delivery.

A bolus LVD injector device is configured to deliver a bolus of the respective medicament to bring a volume of the medicament into a patient's body within a predetermined time. The injection rate, however, may not be critical, i.e., tight control may not be necessary. However, there may be an upper (physiological) limit to the delivery rate in order to avoid damage to the tissue surrounding the delivery site. The time taken to deliver a bolus dose of medicament may be between a few minutes and many hours depending on a number of factors including the quantity (volume) of medicament, the viscosity of the medicament and the nature of the injection site at which the injection device is intended to be used.

From a user or health care professional perspective, it is desirable for an injection device to be configured to minimally impact the patient's lifestyle and schedule, providing the patient with minimal reminder of his or her disease between the injections. The treatment schedule for therapies is usually intermittent, i.e. may be one injection per week, one injection every other week, or one per month. Therefore, the patient usually has no routine in dealing with his or her disease, and hence has minimal routine/experience in performing the required injections. Thus, configuration of the injection device to simplify its operation by patients is highly desirable.

If an LVD is intended for bolus operation, the configuration of the LVD injection device is quite different compared to an LVD injection device that is intended to be used for basal operation. Also, its use is quite different. For instance, a basal-type insulin pump generally is relatively expensive as it includes many sophisticated diabetes specific features like programmable delivery rate profiles, bolus calculators etc. Further, the connection to the body via an infusion set allows the patient to handle and manipulate the pump in his/her field of view while the therapy is ongoing. Further, diabetes patients usually have a routine in setting-up the infusion set, connecting and operating the pump, and disconnecting the pump temporarily for events like taking a shower so not to expose the pump to water. In contrast, the bolus injector devices described above can be relatively simple and inexpensive devices. They may be provided as single-use devices, which cannot be recharged with medicament, which further reduces complexity and cost.

To use an LVD injection device, it is first located on a suitable injection site on a patient's skin. The device is typically adhered to the patient's skin throughout the medicament delivery process. Injection is usually initiated by the patient or another person (user). Typically, the initiation is started by a user operation, such as depressing a switch (mechanical or electrical) or by placing the LVD on the patient's body and depressing a lever on the device's underside. If the LVD includes electronics, a controller can operate the device. Operation includes firstly injecting a needle into the user and then causing the injection of medicament into the user's tissue. The delivery process can take several minutes up to several hours. Following, the LVD can be removed from the injection site and disposed of.

Biological medicaments are being increasingly developed which comprise higher viscosity injectable liquids and which are to be administered in larger volumes than long-known liquid medicaments. LVDs for administering such biological medicaments may comprise a pre-filled disposable medicament delivery device or, alternatively, a disposable medicament delivery device into which a patient or medical personnel must insert a drug cartridge prior to use.

The terms “drug”, “medicament”, or “pharmaceutical”, which are used interchangeably herein, mean a pharmaceutical formulation that includes at least one pharmaceutically active compound, which may be, for example, a small molecule or biologic active pharmaceutical ingredient. Further descriptions of contemplated drugs, medicaments or pharmaceuticals are provided below.

Standards or best practices for the design of medicament delivery devices may require or recommend providing an unobstructed path from the medicament container to a needle or cannula used to deliver the medicament to a user. Additionally, standards or best practices for manufacturing and assembling a medicament delivery device, such as a large volume or reservoir-type injection device, may require that certain steps in the assembly occur in a clean room or aseptic facility or may require sanitizing certain components. Embodiments may provide an aseptic sterile path from a drug container to the needle or cannula, according to the present disclosure, without requiring that all aspects of the assembly be performed in clean room or highly sterilized environments. Embodiments may provide flexibility and cost-savings in manufacturing by allowing aseptic conditions to be maintained in the device without requiring the use of a clean room or other highly sanitized environment during all steps of the manufacturing process.

In some embodiments, the present disclosure may be directed to a large volume delivery device that includes an external housing configured to be held against the user's body when the device is in use, a medicament container, a fluid pathway connected to the medicament container and extending to an insertion mechanism, such as a needle insertion mechanism or a trocar and cannula insertion mechanism.

In some embodiments, the present disclosure may be directed toward a drive mechanism for the large volume device that can force the medicament from the medicament container. In some embodiments, the drive mechanism may include a piston pump or a plunger pump, and may be activated by the user by applying force onto a button sled that interacts with the drive mechanism, or by a separate piston or plunger. In some embodiments, the piston pump or the plunger pump may be driven by one or more springs, drive screws, motors, or any other suitable drive mechanism.

In some embodiments, the drive mechanism may include a drive housing including one or more ridges and protrusion(s), a drive release latch, a piston, and a drive outer sleeve.

In some embodiments, the drive mechanism may interact with an end of dose (EOD) switch, which may be adopted by the large volume device.

In some embodiments, the present disclosure may be directed toward a method of forming an operation flow by activating the drive mechanism.

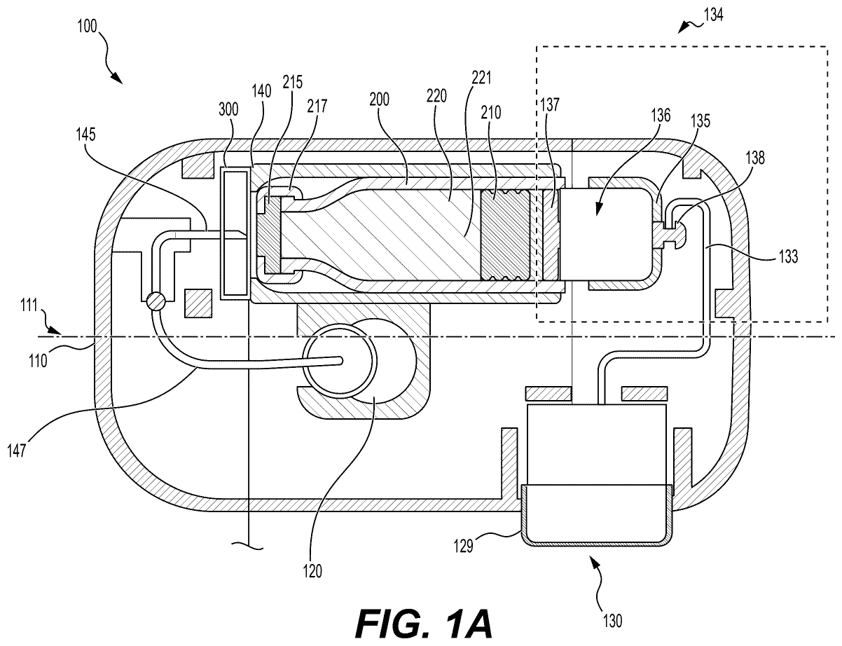

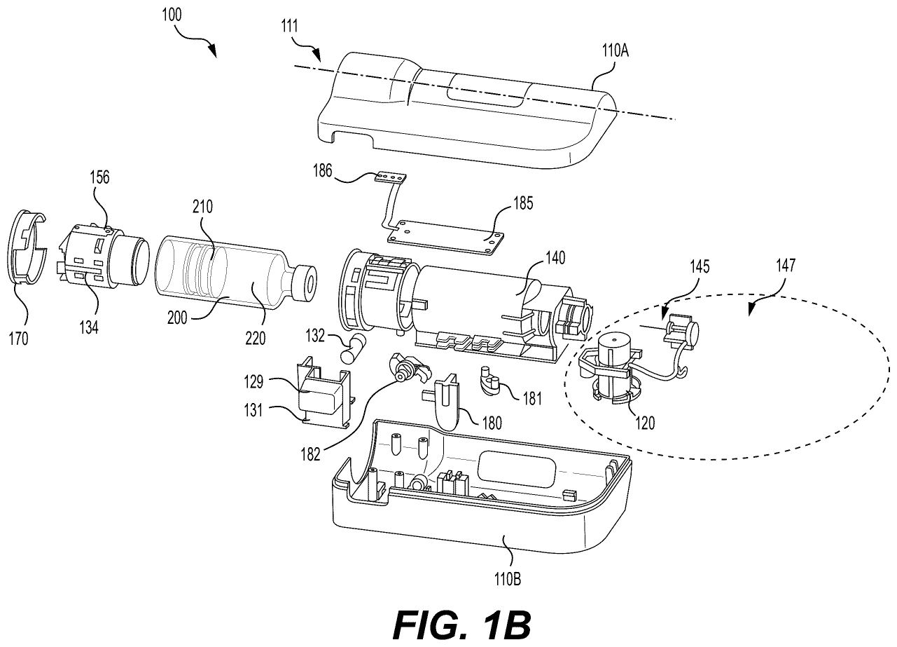

FIG. 1A illustrates an exemplary large volume delivery device 100 with an external housing 110, a needle insertion mechanism 120, a release mechanism 130, a drive mechanism 134, a medicament container sled 140, a needle insertion mechanism 120, and a fluid path 147. The release mechanism 130 includes a button 129, a button sled 131 (shown in FIG. 1B) and a button biasing member or other power source (not shown). The button 129 can be connected to the button sled 131. In some embodiments, the button 129 is part of the button sled 131. The drive mechanism 134 includes a drive housing 135, a drive release latch 133, a piston mechanism including a piston 137, a drive outer sleeve 136, and a detent 138.

A medicament container 200 is included within the large volume delivery device 100. The medicament container 200 includes a plunger 210, a stopper 215 (which may alternatively be referred to as a septum), and a cap 217 (e.g., a crimp cap) and has an internal volume 220 at least partially filled with a medicament 221. The container 200 may be, for example, a glass vial with a polymer plunger and septum. Generally, the container 200 will include a standard medicament container such that the disclosed devices and fluid path connections can be use with existing, standard containers without the need for development of a specialized container or medicament cartridge. The plunger 210 may be driven by the piston 137.

FIG. 1 should be understood to be an exemplary device. The drive mechanism 134 can include one or more springs, but other drive mechanisms or power sources may be possible. Other drive mechanisms may include other biasing elements, screw drives, gear drives, gas or chemical sources, or electric motors. The medicament container 200 should be understood as exemplary only. For example, different types of containers or vials may be used, including containers with different stopper or piston arrangements, or without a stopper or piston.

The large volume delivery device 100 is prepared for use by loading the medicament container 200 and setting the drive mechanism 134 in the energized state (as shown in FIG. 1). The drive outer sleeve 136 is held in an energized state by engagement of the drive release latch 133 with a detent 138 of the piston 137. To operate the device 100, the device 100 is positioned with the needle insertion mechanism 120 against skin of a user. The device 100 may be attached to skin of the user by removing a removable covering mounted on the external housing 110 to expose an adhesive on the external housing 110. The user, or someone assisting the user, presses and shifts the button 129 and/or the button sled 131 of the release mechanism 130, so that the drive release latch 133 is shifted out of the detent 138 and release the drive outer sleeve 136, whereupon the drive outer sleeve 136 drives the piston 137 against the plunger 210 of the medicament container 200, forcing the plunger 210 into the medicament container 200 and pressurizing the internal volume 220 with medicament 221. An action of the device 100 causes the needle 145 to pierce the stopper 215 and access the medicament 221 within the medicament container 200. The pressurized medicament flows from the medicament container 200 into the needle 145 and then along the fluid path 147 to the needle insertion mechanism 120. An action of the device 100 causes the needle insertion mechanism 120 to insert a needle and/or cannula into the user. The medicament 221 is delivered through the needle or cannula to the patient.

The device 100 also includes a fluid path connection 300. The fluid path connection 300 is positioned near the stopper 215 and needle 145 and permits the needle 145 to travel through an unobstructed path to the stopper 215 to establish a fluid path connection with the medicament container 200. In some embodiments, the fluid path connection 300 is connection is configured to isolate the fluid path during storage and/or use and to maintain an aseptic or sterile fluid path.

FIG. 1B illustrates the exemplary large volume delivery device 100 and its assembly from a perspective view. As shown in FIG. 1B, the exemplary device 100 includes the external housing 110 that includes the medicament container sled 140 and a drive holder 160, a medicament container 200 that includes a plunger 210 and an internal volume 220 to hold medicament, the drive mechanism 134, an end of dose (EOD) switch assembly 156, and a housing drive cap 170.

As shown in FIG. 1B, the external housing 110 of the exemplary device 100 further includes a top cover 110A and a bottom cover 110B, an interlock fin 180, and interlock with an interlock spring 182, the release mechanism 130 including a button 129, a button sled 131 and a button spring 132, and a needle insertion mechanism (NIM) lever 181 and a fluid path assembly 147. Additionally, the housing 110 includes an axis 111, along which the medicament container 200 moves via movement of the medicament container sled 140.

In some embodiments, the medicament container 200 may be configured to enter the medicament container sled 140, and may be configured to retained into the medicament container sled 140 with a secure connection. For example, the container may be secured by at least at least one snap-fit connector on the medicament container sled 140 or by a friction fit or other connection.

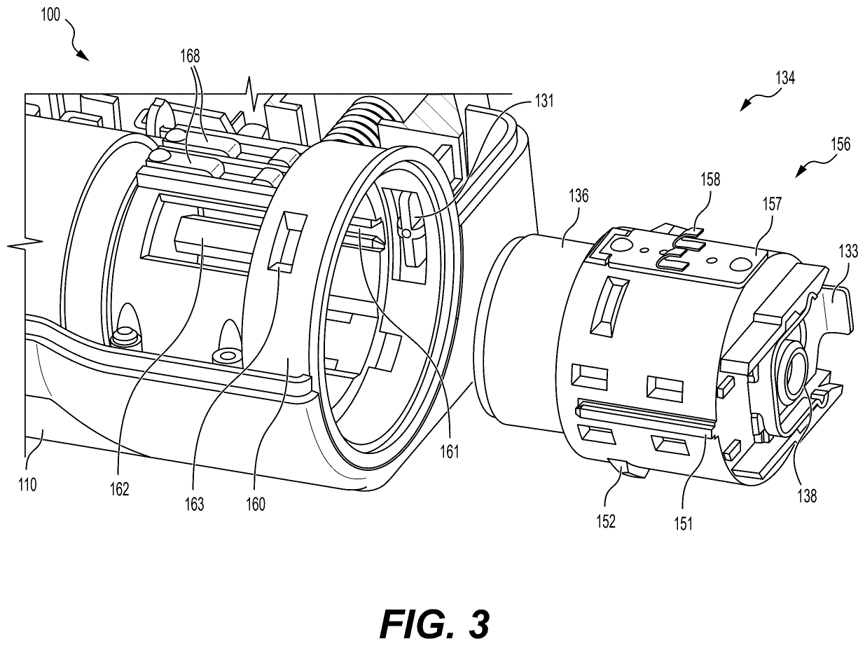

In some embodiments, the drive mechanism 134 may be configured to enter the drive holder 160, and may be configure to be retained into the drive holder 160. For example, the drive mechanism may be secured by at least one snap fit connector on the drive holder 160 and/or at least one mechanical stop. More details on the connection between the drive mechanism 134 and drive holder 160 will be described with respect to FIG. 3 and accompanying descriptions.

In some embodiments, the drive mechanism 134 may be configured to interact with the EOD switch assembly 156 to detect nearing of end of dose.

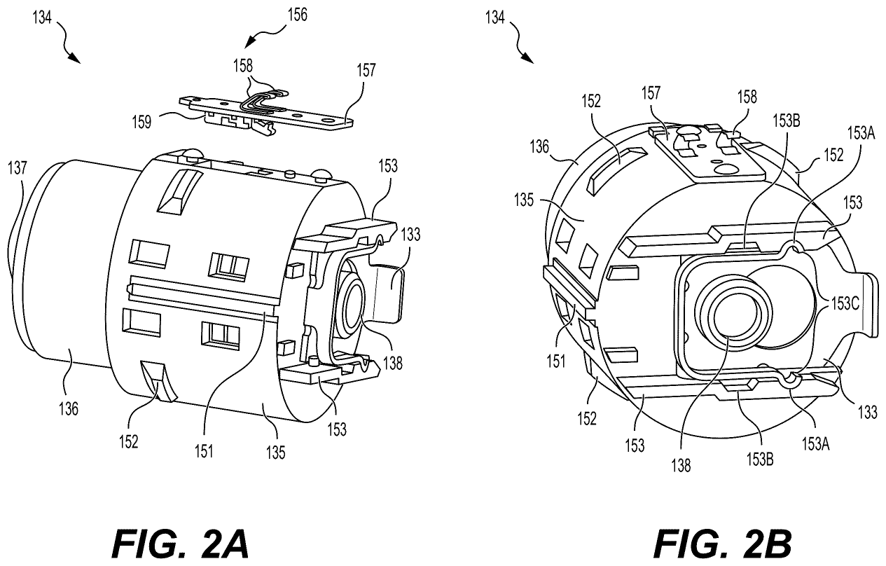

FIGS. 2A and 2B are perspective views illustrating the drive mechanism 134 in accordance with one or more embodiments. As shown in FIGS. 2A and 2B, the drive mechanism 134 may include the drive housing 135, the drive release latch 133, and a piston 137. In some embodiments, the drive mechanism 134 is configured to fit into a drive holder fixed on the external housing 110 of the device 100.

In some embodiments, the drive housing 135 may be configured to roughly have a cylindrical shape. In some embodiments, the drive housing 135 may include at least one ridge 152 and at least one protrusion 151 on an outer lateral surface of the drive housing 135. In some embodiments, the drive housing 135 may include a pair of walls 153 on an outer bottom surface of the drive housing 135. The at least one ridge 152, the at least one protrusion 151, and the pair of walls 153 may each have at least one flat surface that perpendicular to outer surface of the drive housing 135.

In some embodiments, the drive housing 135 may include four ridges 152. In some embodiments, the drive housing 135 may include one or two protrusions 151.

In some embodiments, the pair of walls 153 may form a pair of first lock grooves 153A and a pair of second lock grooves 153B. The walls 153 may be configured to interact with the drive release latch 133 through the first lock grooves 152A and the second lock grooves 153B. In some embodiments, the first lock grooves 153A may hold the drive release latch 133 at an initial position. In some embodiments, the second lock grooves 153B may hold the drive release latch at an activated position.

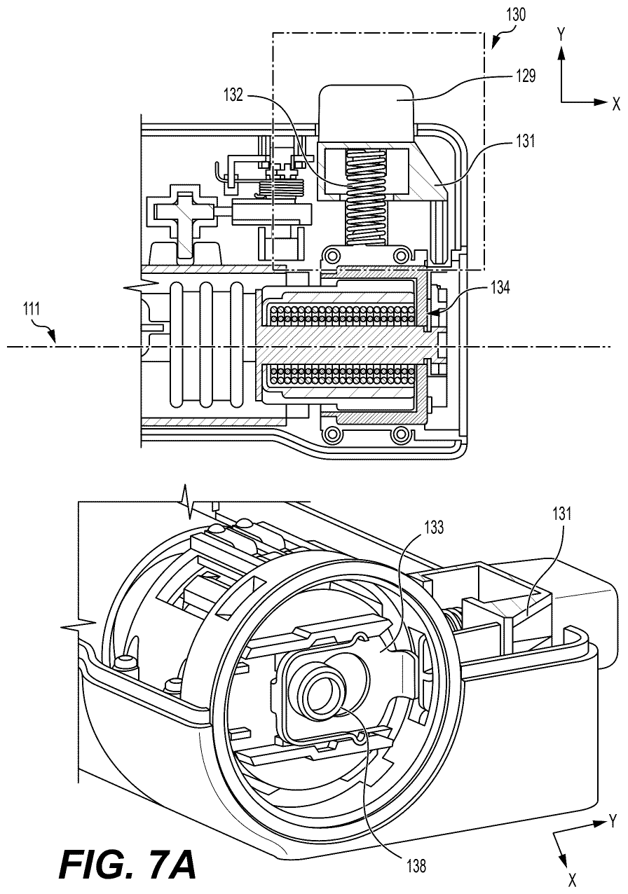

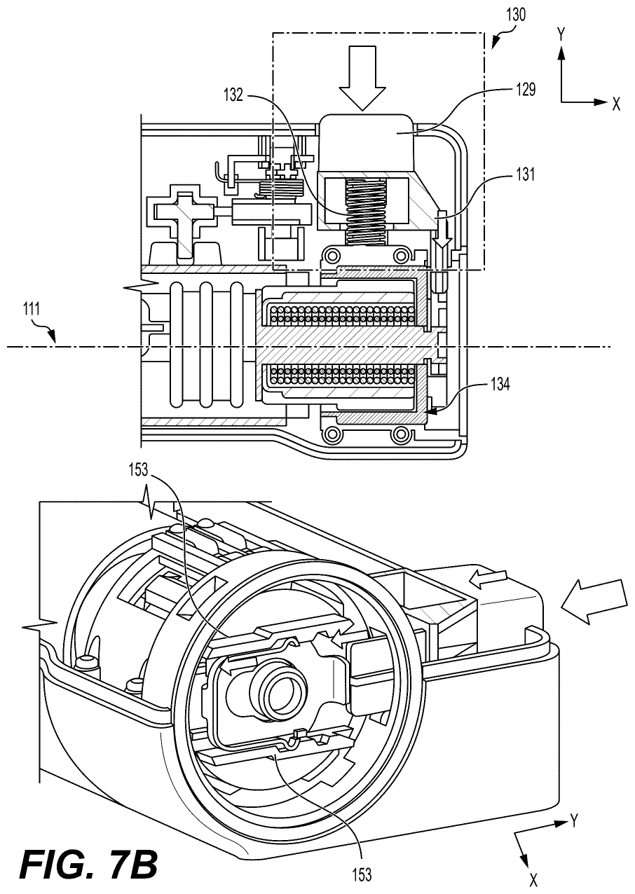

In some embodiments, the drive release latch 133 may include an irregularly shaped hole and a pair of lock protrusions 153C. The drive release latch 133 may be configured to move between and the initial position and the activated position along the pair of walls 153. In some embodiments, the drive release latch 133 may be configured to be maintained at the initial position where holds the drive mechanism in an energized state by engaging with the detent 138 of the piston 137. At this initial position, the lock protrusions 153C are locked between the first lock grooves 153A. In some embodiments, the drive release latch 133 may be configured to face a feature of a button sled through a window in the drive holder. In some embodiments, the drive release latch 133 may be configured to be pushed from the initial position to the activated position, and to release the piston 137 upon a user applying pressure on the button 129 and/or the button sled 131. The piston 137 is released when the drive release latch is locked between the second lock grooves 153B at the activated position. More details in these regards will be described in FIGS. 7A and 7B and the accompanying description.

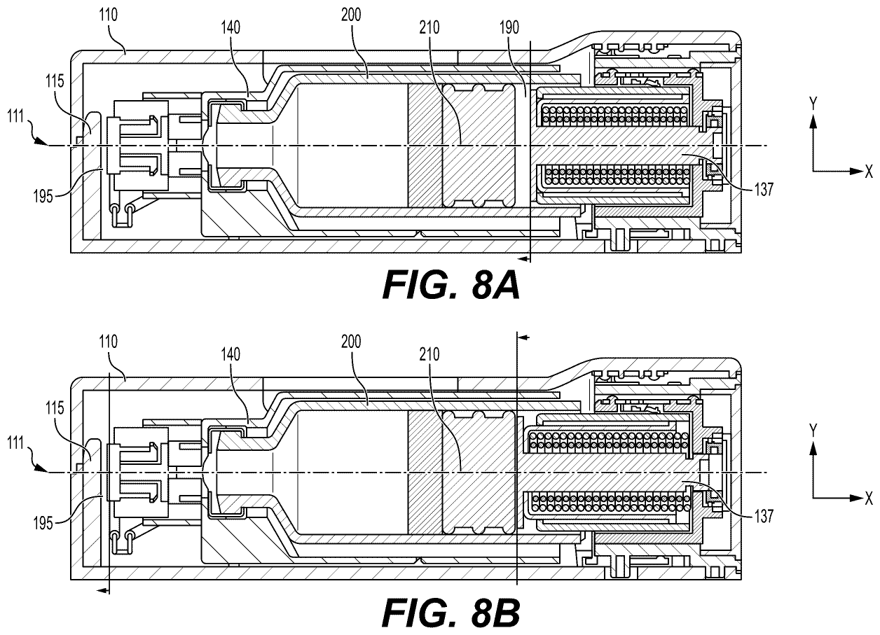

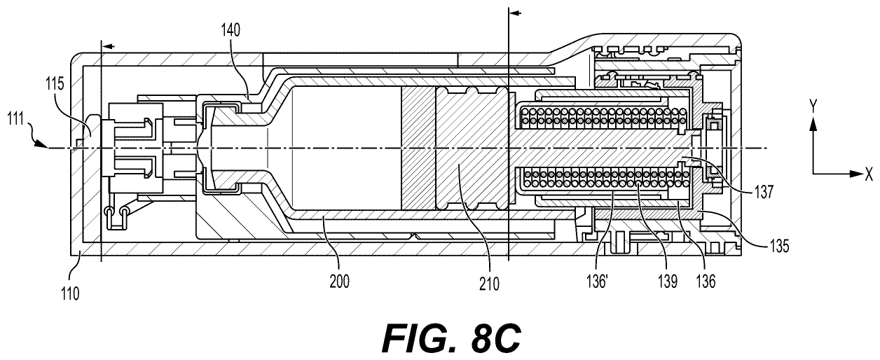

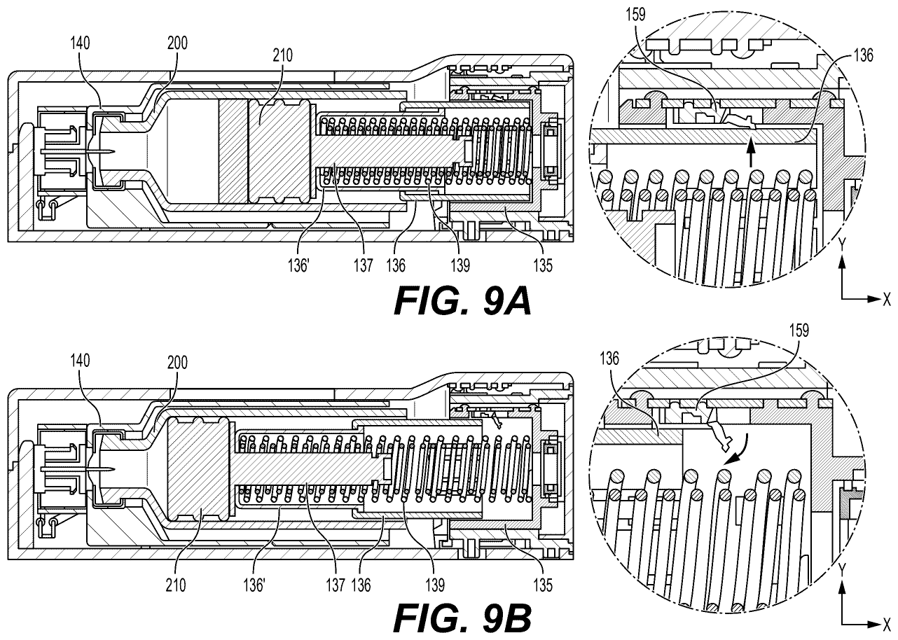

In some embodiments, the piston 137 may include in a drive inner sleeve (not shown in FIGS. 2A and 2B). In some embodiment, the piston 137 may be configured to move forward along an X-axis of the drive mechanism 134, which is same as the axis 111 of the external housing 110. In some embodiments, the piston 137 may be driven by a spring (not shown in FIGS. 2A and 2B). In some embodiments, the piston 137, the drive inner sleeve, the drive outer sleeve 136, the drive housing 135 and the spring may be configured together to form a telescopic mechanism. More details of the telescopic mechanism will be described in FIGS. 8A-9B and the accompanying description.

In some embodiments, the drive mechanism may be configured to interact with the EOD switch assembly 156. In some embodiments, the EOD switch assembly 156 may include an EOD switch printed circuit board assembly (PCBA) 157, an EOD switch 159, and a pair of spring contacts 158. In some embodiments, the EOD switch PCBA 157 may be heat staked to the drive housing 135 and may include electrical interface with device electronics. In some embodiments, the EOD switch 159 may be mounted on the EOD switch PCBA 157 and may be configured to interact with the drive outer sleeve 136. In some embodiments, the pair of spring contacts 158 may be configured to slide against a drive holder contacts (not shown in FIGS. 2A and 2B) to connect the EOD switch PCBA 157 to the drive holder of the large volume delivery device 100, wherein the drive holder contacts may be heat staked on the drive holder. In some embodiments, the drive mechanism and the switch assembly 156 may be configured to interact and to release the EOD switch upon detecting when the delivery device 100 is nearing an end of desired dose prior to when an end of dose occurs.

FIG. 3 is a perspective view illustrating an assembly in accordance with one or more embodiments shows an assembly of the drive mechanism 134 from one perspective. As shown in FIG. 3, a drive holder 160 is fixed in the external housing 110 of the large volume delivery device 100, for example, by a plurality of screws. In some embodiments, the drive holder 160 may include at least one groove 161, at least one snap-fit 162, and at least one snap hole 163. In some embodiments, the drive holder 160 may include a pair of drive holder contacts 168, where the drive holder contracts 168 may be heat staked on the drive holder 160.

In some embodiments, the drive holder 160 may include four snap-fits 162. In some embodiments, the drive holder 160 may include one or two grooves 161. In some embodiments, the drive holder 160 may include four snap holes 163.

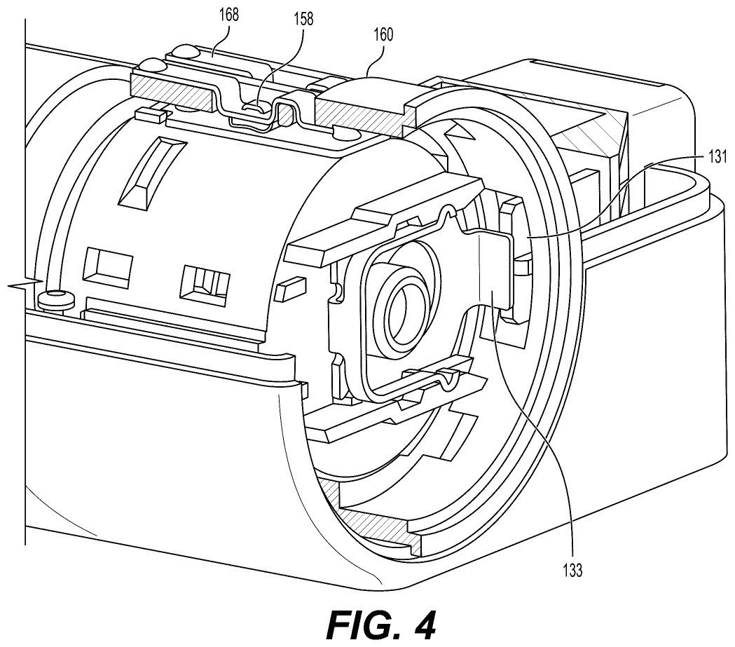

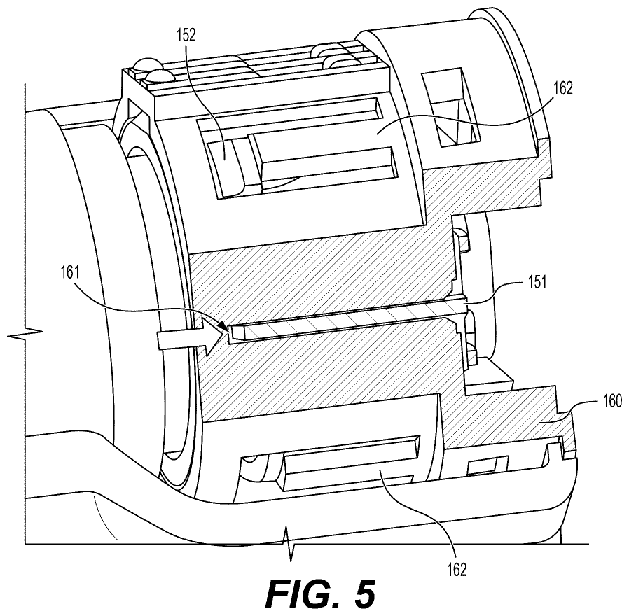

In some embodiments, the drive mechanism 134, the EOD switch assembly 156, and the drive holder 160 may be configured to form an assembly. For example, the EOD switch assembly 156 may be configured to be fit onto the drive mechanism 134. Further, the drive mechanism 134 with the fitted EOD switch assembly 156 may be configured to be inserted into and be retained in the drive holder 160. Specifically, during the insertion, the pair of spring contacts 158 slide against the drive holder contacts 168 and connect the EOD switch PCBA 157 to the drive holder 160 (as shown in FIG. 4). In addition, during the insertion, the at least one ridge 152 is snapped into the at least one snap-fit 162, and the at least one protrusion 151 slides into the at least one groove 161 to form mechanical stops. As such, the drive mechanism 134 with the EOD switch assembly 156 is retained in the drive holder 160 by the formed mechanical stops(as shown in FIG. 5).

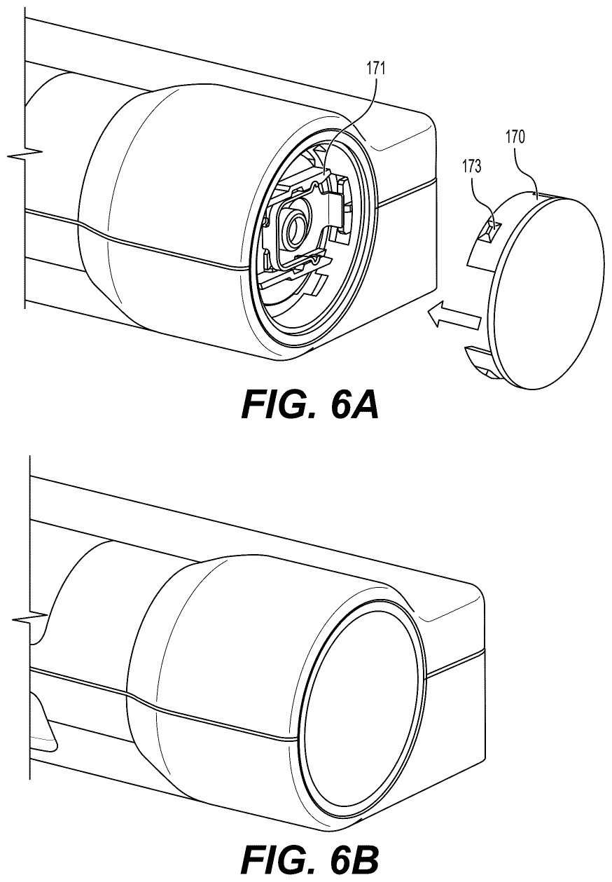

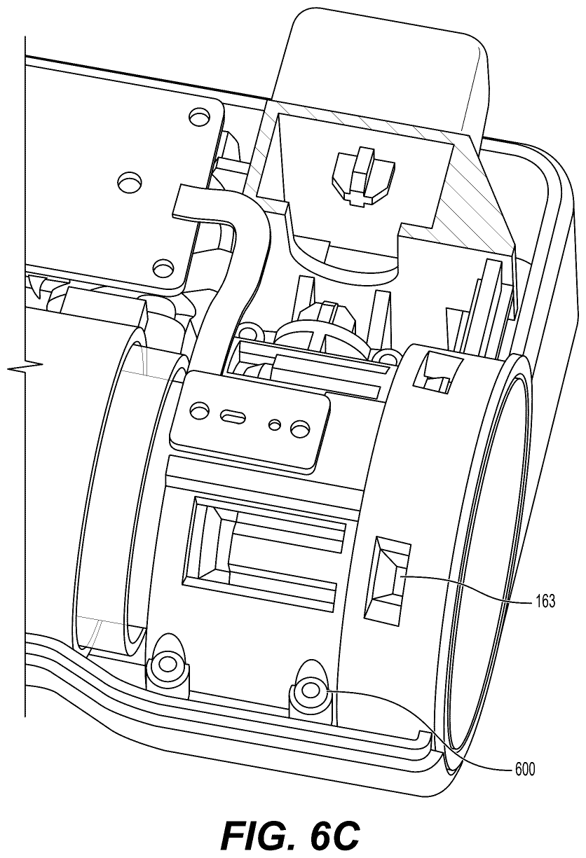

FIGS. 6A-6C are perspective views illustrating an assembly in accordance with one or more embodiments. As shown in FIG. 6A, upon that the drive mechanism 134 is inserted and retained in the drive holder 160, a housing drive cap 170 may be snapped into the drive holder 160 to close the large volume delivery device 100. In some embodiments, the housing drive cap 170 may be designed with no external grips or assembly features to avoid being disassembled (as shown in FIG. 6B). In some embodiments, the housing drive cap 170 may include at least one cap ridge 173 that are configured to fit into the at least one snap hole 163 of the drive holder 160. As shown in FIG. 6C, upon snapping the housing drive cap 170 into the drive holder 160, the at least one cap ridge 173 is snapped into the at least one snap hole 163 to form a closed device assembly. In some embodiments, the housing drive cap 170 may include four cap ridges 173.

In various embodiments, the drive holder 160 is configured to securely retain the drive mechanism such that the drive mechanism 134 can exert significant force on the plunger 210 of the medicament container 200, thereby allowing the medicament container sled 140 to slide forward to establish a fluid connection with the fluid path 147 as the needle 145 pierces the medicament container 200. Importantly, the drive holder 160 can be secured in place while placed adjacent the cartridge sled 140, with the medicament container sled 140 being able to move axially along a long axis of the large volume delivery device 100. The drive holder 160 may be secured using one or more of a threaded connection 600, a snap-fit connection, a friction fit, or a mechanical stop (e.g., a mechanical protrusion in the external housing). As such, the drive mechanism is held in place while the medicament container sled 140 is free to move.

During assembly, the various components, including the medicament container sled 140 and drive holder 160, as well as all components of the fluid path 147 can be loaded into a bottom cover 110B, by placing them into the bottom cover 110B, and the top cover 110A can be positioned to allow closing of the device 100.

Later, sterilized or aseptic components including the medicament container 200 can be loaded through a rear opening 171 by axial loading. In addition, the drive holder 160, having already been secured in place, can be loaded with the drive mechanism 134 via axial loading and closure of the housing drive cap 170.

FIGS. 7A and 7B are perspective views illustrating activation of the drive mechanism 134 in accordance with one or more embodiments. As shown in FIG. 7A, upon that the drive mechanism 134 is inserted and retained in the drive holder 160, the drive release latch 133 may contact with a button 129 and/or a button sled 131 of the release mechanism 130. In some embodiments, the drive release latch 133 may be released and move along the pair of walls 153 to unlock the detent 138 upon receiving pressure from the button 129 and/or the button sled 131. Specifically, as shown in FIG. 7B, pressure is applied to the button 129 and/or the button sled 131 of the release mechanism 130, for example, by the user, the button 129 and/or the button sled 131 is shifted to move along an Y-axis (which is perpendicular to the axis 111 of the external housing 110) and pushes the drive release latch 133 to move along between the pair of walls 153 from the initial position (shown in FIG. 7A) to the activated position (shown in FIG. 7B) to release the detent 138. Once the detent 138 is released by the drive release latch 133, the piston 137 is released.

FIGS. 8A-8C are perspective views illustrating an operation flow in accordance with one or more embodiments. As shown in FIG. 8A, the piston 137 is just released upon that the user presses the button 129 and/or the button sled 131. In some embodiments, a gap 190 may exist between the plunger 210 of the medicament container 200 and the piston 137 at this initial state. As shown in FIG. 8B, upon being released, the piston 137 moves forward along the axis 111 of the external housing 110 to recover the gap 190 until it contacts the plunger 210. As shown in FIG. 8C, in some embodiments, the piston 137 may continue to move forward along the axis 111 of the external housing 110 to push the plunger 210 and the medicament container sled 140 to move forward, until a gap 195 between the medicament container sled 140 and bottom cover ribs 115 of the external housing 110 is recovered.

FIGS. 9A and 9B are perspective views illustrating activation of an EOD switch mechanism in accordance with one or more embodiments. As shown in FIG. 9A, the piston 137 continues to push the plunger 210 to move forward along the axis 111 of the external housing 110. As shown in FIGS. 8A-9B, the piston 137, the drive inner sleeve 136′, the device outer sleeve 136, the drive housing 135, and spring 139 may be configured to form a telescopic mechanism. In some embodiments, as the piston 137 moves forward along the axis 111 of the external housing 110 under extension force of the spring 139, the drive inner sleeve 136′ interacts with and pulls the drive outer sleeve 136 to move forward along. As shown in FIG. 9A, the EOD switch 159 is kept in an open state by the drive outer sleeve 136.

FIG. 9B shows a state of nearing end of dose, where the plunger 210 almost reaches the end of the medicament container 200 and the injection is almost completed. In some embodiments, the drive outer sleeve 136 may be configured so that upon nearing end of dose, the drive outer sleeve 136 reaches a position where it just releases the EOD switch 159. In some embodiments, when the EOD switch 159 is released by the drive outer sleeve 136 upon nearing end of dose, the spring 139 may be fully released.

In some embodiments, when the detection of the nearing end of dose occurs and when the EOD switch 159 is released, the piston may reach partially, for example, 85%-95% of its maximum position of fully expended. In some embodiments, the EOD switch 159 may be configured to detect the nearing end of dose and be released when a volume of remaining medicament is anywhere between 5%-20% (5%, 15%, or 20%) of a full dose of medicament. In some embodiment, the EOD switch 159 may be configured to detect the nearing of end of dose and be released when remaining injection time is any time between 10 seconds to a minute.

### Exemplary Drugs or Medicaments

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA (including RNAi & siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a medicament delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In other examples, the container may be made of a flexible elastomeric material and designed to be loaded by the health care provider or patient, then placed on the body for administration. In some instances, the chamber may be designed to store a drug for about 1 month to about 2 years. Storage may occur at room temperature (e.g., about 20° C.), or refrigerated temperatures (e.g., from about −4°C to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the medicament delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codable amino acids, or amino acids, including non-codable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin;

B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a polysulphated form of the above-mentioned polysaccharides, and/or a pharmaceutically acceptable salt thereof.

An example of a pharmaceutically acceptable salt of a polysulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti-IL-6 mAb (e.g., Sarilumab), and anti-IL-4 mAb (e.g., Dupilumab).

Pharmaceutically acceptable salts of any API described herein are also contemplated for use in a drug or medicament in a medicament delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. A system for delivering medicament, comprising:
an external housing;
a medicament container sled within the external housing;
a drive holder;
a medicament container comprising a plunger and an internal volume to hold medicament; and
a drive mechanism;
wherein the drive mechanism is configured to enter the driver holder and to be securely retained in the drive holder.

2. The system of claim 1, wherein the drive mechanism comprises:
a drive housing;
a drive release latch, and
a piston mechanism.

3. The system of claim 1, wherein the medicament container is configured to enter the medicament container sled and to be retained in the medicament container sled via a snap-fit mechanism on the medicament container sled.

4. The system of claim 1, wherein the medicament container sled is configured to move along a long axis of the system to engage the medicament container with a fluid path connector.

5. The system of claim 1, wherein the drive holder is configured to engage the drive mechanism via a snap-fit connection.

6. The system of claim 1, wherein the drive holder is secured within the external housing with at least one of a threaded connector, a snap-fit connection, a friction-fit, a mechanical stop, or a welded connection.

7. The system of claim 1, wherein the external housing comprises a long axis and a rear opening, and wherein the medicament container sled and drive holder are aligned with the drive housing and the rear opening such that the medicament container and drive mechanism can be inserted through the rear opening to assemble the system.

8. The system of claim 2, wherein the piston mechanism comprises comprising a drive outer sleeve and a detent.

9. The system of claim 1, further comprising and end-of-does (EOD) switch assembly comprising an EOD switch and an EOD switch printed circuit board assembly (PCBA), wherein the EOD switch is mounted on the EOD switch PCBA.

10. The system of claim 1, wherein the external housing comprises:
a top cover;
a bottom cover; and
an end cap.

11. A drive mechanism, comprising:
a drive housing, wherein the drive housing comprises at least one ridge, at least one protrusion, and a pair of walls,
a drive release latch, and
a piston mechanism comprising a piston, a drive outer sleeve, and a detent,
wherein the pair of walls are configured to interact with the drive release latch, and
wherein the drive mechanism is configured to fit into a drive holder of an external housing.

12. The drive mechanism of claim 11, further comprising:
an end of dose (EOD) switch assembly.

13. The drive mechanism of claim 11, wherein the drive holder comprises at least one snap-fit connector for securing the drive mechanism.

14. The drive mechanism of claim 13, wherein, upon inserting the drive mechanism into the drive holder, the drive mechanism is retained in the drive holder by mechanical stops formed by snapping the at least one ridge into the at least one snap-fit and by sliding the at least one protrusion into the at least one groove.

15. The drive mechanism of claim 11, wherein the pair of walls comprises first lock grooves and a second lock grooves, wherein the first lock grooves hold the drive release latch at an initial position, wherein the second lock grooves hold the drive release latch at an activated position.

16. The drive mechanism of claim 15, wherein the drive release latch faces a feature of a button sled through a window in the drive holder.

17. The drive mechanism of claim 16, wherein the drive release latch is configured to release the piston upon applying pressure onto the button sled.

18. The drive mechanism of claim 17, wherein upon applying the pressure onto the button sled, the drive release latch moves from the initial position to the activated position.

19. The drive mechanism of claim 18, wherein the piston is released when the drive release latch is at the activated position.

20. The drive mechanism of claim 17, wherein the piston is configured to move forward along an axis of the drive mechanism.

21. The drive mechanism of claim 20, wherein upon release, the piston moves forward along the axis to recover a gap between the piston and a plunger of a medicament container.

22. The drive mechanism of claim 17, wherein the piston is configured to push a medicament container sled along the axis by interacting with the plunger.

23. The drive mechanism of claim 12, wherein the drive mechanism is configured to release the EOD switch assembly upon detecting when a delivery device is nearing an end of desired dose prior to when an end of dose occurs.
